(19) United States
(12) Patent Application Publication
(10) Pub. No.: US 2025/0157627 A1
(43) Pub. Date: May 15, 2025

(54) SYSTEMS AND METHODS FOR DIGITAL ENGAGEMENT AND HEALTH BIOMETRIC ANALOG DATA COLLECTION FOR PATIENTS WITH DEMENTIA

(71) Applicant: Mentia Health, Inc., Elk Grove, CA (US)

(72) Inventors: Mandy Salomon, Elk Grove, CA (US); Serge Soudoplatoff, Elk Grove, CA (US); Paul Fentress, Elk Grove, CA (US); Ilia Vialikov, Elk Grove, CA (US); Zhonqiao Lin, Elk Grove, CA (US)

(73) Assignee: Mentia Health, Inc., Elk Grove, CA (US)

(21) Appl. No.: 18/947,632

(22) Filed: Nov. 14, 2024

Related U.S. Application Data
(60) Provisional application No. 63/548,456, filed on Nov. 14, 2023.
(60) Provisional application No. 63/555,651, filed on Feb. 20, 2024.

(51) Int. Cl. G16H 20/70; A61B 5/00; A61B 5/16; G06T 13/40; G16H 10/60; G16H 50/20

(52) U.S. Cl. CPC G16H 20/70; A61B 5/0077; A61B 5/165; A61B 5/4088; A61B 5/4803; A61B 5/7275; A61B 5/746; G06T 13/40; G16H 10/60; G16H 50/20; G06T 2210/41

(57) ABSTRACT

In an embodiment, a system for digital engagement is disclosed that is configured to operate an application for a patient with dementia. The application includes at least one applied virtual environment. The system is further configured to evaluate input of the patient based on a mixed model for observing digital engagement, and to engage the patient with the application using dialogue delivered by at least one virtual avatar, the virtual avatar being supported by artificial intelligence and the dialogue being determined based on the evaluated input. The system is further configured to log and evaluate progress data of the patient and to provide feedback and support based on the evaluated progress data.

### CROSS REFERENCE TO RELATED APPLICATIONS

This application claims the priority of the following applications, which are incorporated by reference herein in their entirety:
U.S. Provisional Application No. 63/548,456, entitled “SYSTEMS AND METHODS FOR DIGITAL ENGAGEMENT,” filed on Nov. 14, 2023, and
U.S. Provisional Application No. 63/555,651, entitled “SYSTEM AND METHOD FOR DIGITAL ENGAGEMENT FOR PATIENTS WITH DEMENTIA,” filed on Feb. 20, 2024.

### COPYRIGHT NOTICE

A portion of the disclosure of this patent document contains material, which is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### BACKGROUND

This application relates to a system and method for digital engagement, and in particular, a digital health application for patients with dementia. The digital health application comprises a personalized conversational and interactive system with guided activities built with video game technology, supported by artificial intelligence.

Dementia extends well beyond its pathology. From the point of diagnosis, dementia reverberates through families and extends into communities, influencing societal attitudes on disability and the elderly. In cases where persons with dementia live at home, as the majority do, undiagnosed behaviors add considerable anxiety and stress to others living with them. When home conditions are problematic, numbers of referrals to community mental health teams increase and demand for respite care grows. Furthermore, the collapse of home care systems leads to a rise in hospitalization, administering of antipsychotic drug treatments, and the move to full-time residential care. With 50 million cases worldwide, dementia impacts productivity and challenges healthcare resources. In the United States alone, the number of hours devoted to caring for persons with dementia is estimated at 90 billion hours per year.

In the United States, Medicare alone annually spends $100 billion more for seniors with dementia than for those without dementia. Emergency department (“ED”) visits and ensuing hospital stays are significant factors driving high costs and poor health outcomes for people living with dementia. As the number of persons with dementia is projected to reach 150 million by 2050, a global movement has been gaining traction to bring greater awareness and reduce stigma surrounding dementia, as well as to address the limitations of healthcare resources.

The characteristics of dementia include a progressive loss of functioning in areas such as cognitive thought, memory, speech, movement, concentration, emotions, and judgment. Thus, dementia impacts a person's ability to perform everyday activities. Dementia is easier to diagnose than the underlying neurodegenerative diseases that cause it, and only in a post-mortem can a definitive diagnosis of the underlying disease be made. This uncertainty means that, although persons with dementia and their families know the condition will progress to the terminal “end stage”, they are never sure of the journey of each individual patient because each dementia-causing neurodegenerative disease impacts different parts of the brain and each has its pathology and clinical presentations.

Neurodegenerative diseases such as Alzheimer's disease make up the most common cases of dementia, accounting for 50-60% of all persons with a dementia diagnosis. Typically, Alzheimer's begins with the loss of the episodic memory, characterized by problems with learning and recall. Vascular dementia, the second most common pathology, is a complication of vascular-related events such as strokes. Other neurodivergent diseases such as Frontotemporal Dementia, Dementia in Parkinson's disease, and Dementia with Lewy Bodies each effect different parts of brain and each disease has different effects on patients. Each neurodivergent disease requires individualized care based on individual patients' needs. Although age is the single largest risk factor, dementia can affect people across their adult lifespan.

While drugs may alleviate certain symptoms of dementia, there is currently no cure or significant treatment strategy available. Although drugs that treat neurological conditions may be beneficial, there is a great deal of disquiet about the ready administering of prescription drugs such as antidepressants and antipsychotics as an easy fix for “symptoms”. Medications can intensify behavioral and psychological symptoms of dementia and the severity of symptoms does not necessarily equate a reduced quality of life, suggesting that much more is still to be learnt about the relationship between the two. Tranquillizers can have severe side effects; for example, the increased risk of a stroke. Medications repress symptoms without understanding why. Evidence has shown increased mortality because of improperly monitored drug use. As an alternative to medications, developing positive interaction in relationships with dementia patients has been shown to have positive effects. Taking an individualized approach to assessing and meeting the needs of people with dementia has also been shown to have positive effects.

Patients with dementia often require full-time care as their condition progresses. A worldwide shortage of caregivers, combined with the fact that at-home caregivers often lack dementia-specific care skills, has led to a dementia care crisis. While individual caregivers may be competent in responding to a person's unmet needs, a more holistic approach is to support a patient's social and psychological well-being with therapeutic activities including recreational therapy, diversional therapy, lifestyle activities, activity-based care, social care, enrichment activities, and non-pharmacological interventions. The percentage of seniors with cognitive impairment is rising-more are living longer at home with more severe symptoms, putting pressure on family and informal caregivers who are not well-equipped to support patients through daily life tasks.

As their dementia progresses, patients are less able to identify pain and its location, and the capacity to manage and track the progression of their co- (or multi) morbidities is severely compromised. Health care management is often reactive with patients twice as likely to end up in emergency rooms than older adults without dementia. Once exposed to the stressful, high energy and unfamiliar ED environment and being examined by staff lacking dementia care and communication skills, patients are commonly prescribed psychiatric drugs, undergo unnecessary tests, or are admitted to a hospital. This leads to patients becoming more distressed while their health further declines.

The lives of people with dementia and those close to them can be improved by exploring digital engagement. In the dementia field, engagement is a process that leads to changed affect. Increased engagement and involvement with external stimuli in people with dementia have associations with increased interest, positive emotions, greater awareness, and functional ability, known as “high and positive affect,” whereas lack of engagement has associations with boredom, depression, apathy, loneliness and behavioral problems. Engagement has been understood as the act of being occupied or involved with external stimulus and described as a necessary foundation for the development of non-pharmacological interventions for persons with dementia to reduce boredom and loneliness by increasing interest and positive emotions.

Digital activities have the ability to connect people, extend friendships, and provide life-long learning and various services to enrich the lives of users. Digital activities create a virtual world containing graphical images that evoke place and space and objects or characters situated within it. Participants can interact using a computer device's interface (e.g. touch pad screen, game controller, keyboard/mouse). A virtual “environment” creates a “world” when its narrative elements (actions, events, characters and a setting) tell a story. The transaction between narratives and the audiences that bring them to life occurs through the digital interface, which must be coherent and meaningful to the user. Embedded rich media and personalized content, including audio effects, video, photographs and music enhance the story. Objects contain animation codes or scripts that respond to users' commands; for example, a ball is made to bounce or a bedroom light is switched on/off.

Digital engagement practices are person-centered. A primary aspect of the person-centered approach is to recognize the full spectrum of persons' individual needs, including their psychological, emotional, spiritual and social needs, rather than merely treating the neuropathology. Indeed, there is now a large body of work linking distressed or uncharacteristic behaviors common to people with dementia diagnoses, such as wandering, repetitive verbalizations, anxiety and social withdrawal, as attempts to communicate needs. Person-centered care accepts that there is therapeutic value in having positive interactions between caregivers and care-recipients. A person-centered caregiver listens, empathizes and helps individuals to communicate and engage as their dementia progresses. The strategy also sees value in flexible routines, that, as far as it is possible, incorporates individually tailored activity programs.

Digital engagement provides for further benefits over non-digital activities. For example, coding language is malleable; it can be reconfigured so that underlying stimuli are intact even though objects that embody them change. As a result, an enrichment activity could be deconstructed to expose essential engagement stimuli and then repurposed or repackaged to suit a person or situation better. The software not only provides customized activities but also accommodates all the stimuli in one place and can be reinterpreted in new or preferred ways without draining resources.

The disclosed system and method for digital engagement comprises a digital health application designed with necessary attributes for use by those with early or mild stages to late or severe stages of dementia. Furthermore, the disclosed software enables participants to have an optimal play experience without the assistance or support of a caregiver. The disclosed digital health application thus fills a gap in the market by vastly increasing wellness for people living with dementia, extending in-home dementia-care skills, providing intelligent support, and by helping free up caregiver time.

### SUMMARY

In an embodiment, a system for digital engagement is disclosed. The system comprises a processor coupled to memory, in which the processor is configured to operate an application for a patient with dementia. The application comprises at least one applied virtual environment. The processor is further configured to evaluate input of the patient based on a mixed model for observing digital engagement and to engage the patient with the application using dialogue delivered by at least one virtual avatar. The evaluated input comprises one or more of speech emotion detection, video emotion detection, speech recognition, and user tapping. The virtual avatar is supported by artificial intelligence and the dialogue is determined based on the evaluated input. The processor is further configured to log and evaluate progress data of the patient and to provide feedback and support based on the evaluated progress data.

In some embodiments, the system can further comprise a plurality of agents supported by artificial intelligence. Each of the plurality of agents can be configured to perform a given task and to cooperate with others of the plurality of agents to generate output.

In some embodiments, the input can be evaluated using one or more of speech emotion detection, video emotion detection, speech recognition, and user tapping. The processor can be configured to evaluate the input of the patient and deliver the dialogue in real time. The processor can be further configured to predict a potential emergency based on the evaluated input and trigger an alarm based on the prediction.

In some embodiments, the dialogue can be formulated based on the evaluated input of the patient. The processor can be further configured to tailor the dialogue based on the patient's medical data, a severity of the patient's dementia, the patient's speech capability, and background information regarding the patient.

In certain other embodiments, the processor can be further configured to engage the patient with the application using guided personalized goals. The processor can be further configured to evaluate progress data of the patient including a progression of dementia over a given period of time. In some embodiments, the applied virtual environment can comprise any 3D room including but not limited to a living room, a bedroom, a garden, a barn paddock and pasture, a bathroom, a café, a temple, or a zoo.

According to an embodiment of the present disclosure, a method for digital engagement performed by a processor coupled to memory is disclosed. The method comprises operating an application for a patient with dementia. The application comprises at least one applied virtual environment. The method also comprises evaluating input of the patient based on a mixed model for observing digital engagement and engaging the patient with the application using dialogue delivered by at least one virtual avatar. The virtual avatar is supported by artificial intelligence and the dialogue is determined based on the evaluated input. The method further comprises logging and evaluating progress data of the patient, and providing feedback and support based on the evaluated progress data.

In some embodiments, the method can further comprise performing, by a given one of a plurality of agents supported by artificial intelligence, an assigned task. The method can also comprise cooperating, by the given agent, with others of the plurality of agents to generate output.

In certain embodiments, the step of evaluating input of the patient can comprise evaluating input of the patient using one or more of speech emotion detection, video emotion detection, speech recognition, and user tapping. In certain other embodiments, the method can further comprise evaluating the input of the patient and delivering the dialogue in real time.

In some embodiments, the method can further comprise predicting a potential emergency based on the evaluated input and triggering an alarm based on the prediction. The method can comprise formulating the dialogue based on the evaluated input of the patient. The method can further comprise tailoring the dialogue based on the patient's medical data, a severity of the patient's dementia, the patient's speech capability, and background information regarding the patient.

In some embodiments, the step of engaging the patient with the application can comprise engaging the patient with the application using guided personalized goals. The step of logging and evaluating progress data of the patient can comprise logging and evaluating progress data of the patient including a progression of dementia over a given period of time. The step of operating the application for the patient with dementia, wherein the application comprises at least one applied virtual environment, can comprise operating an application for a patient with dementia. The application can comprise any 3D room including but not limited to a living room, a bedroom, a garden, a barn paddock and pasture, a bathroom, a café, a temple, or a zoo.

The foregoing summary is illustrative only and is not intended to be in any way limiting. These and other illustrative embodiments include, without limitation, apparatus, systems, methods and computer-readable storage media. In addition to the illustrative aspects, embodiments, and features described above, further aspects, embodiments, and features will become apparent by reference to the drawings and the following detailed description.

### BRIEF DESCRIPTION OF THE DRAWINGS

The invention is illustrated in the figures of the accompanying drawings which are meant to be exemplary and not limiting, in which like references are intended to refer to like or corresponding parts.

FIG. 1 is a diagram of components of a digital health application according to embodiments of the present invention.

FIG. 2 is a diagram of components of a digital health application according to embodiments of the present invention.

FIG. 3 is a diagram of components of a dialogue model of a digital health application according to embodiments of the present invention.

FIG. 4 is a flow diagram of an integrated model architecture of a digital health application according to embodiments of the present invention.

FIGS. 5A-5B are a flow diagram of an integrated model architecture of a digital health application according to embodiments of the present invention.

FIG. 6 is a technology overview flow diagram of a digital health application according to embodiments of the present invention.

FIG. 7 is a flow diagram of a participant interacting with an integrated model architecture of a digital health application according to embodiments of the present invention.

FIG. 8 is a flow diagram of a comprehensive model of digital engagement adapted in some embodiments of the present invention.

FIG. 9 is a flow diagram of how and where a digital app fits into the “Living well with dementia” digital ecosystem in accordance with embodiments of the invention;

FIG. 10 is a flow diagram of framework of engagement in accordance with embodiments of the present invention.

### DETAILED DESCRIPTION OF THE INVENTION

Subject matter will now be described more fully hereinafter with reference to the accompanying drawings, which form a part hereof, and which show, by way of illustration, exemplary embodiments in which the invention may be practiced. Subject matter may, however, be embodied in a variety of different forms and, therefore, covered or claimed subject matter is intended to be construed as not being limited to any example embodiments set forth herein; example embodiments are provided merely to be illustrative. It is to be understood that other embodiments may be utilized and structural changes may be made without departing from the scope of the illustrative embodiments. Likewise, a reasonably broad scope for claimed or covered subject matter is intended. Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of exemplary embodiments in whole or in part. Among other things, for example, subject matter may be embodied as methods, devices, components, or systems. Accordingly, embodiments may, for example, take the form of hardware, software, firmware or any combination thereof (other than software per se). The following detailed description is, therefore, not intended to be taken in a limiting sense.

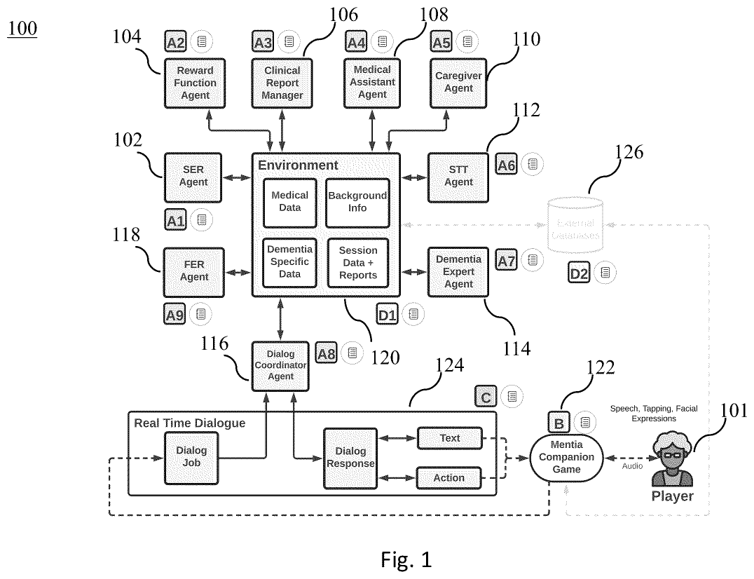

With reference to FIGS. 1-10, a system and method for digital engagement are disclosed that utilize software specifically designed for use by people living with dementia (“PLWD”), or people with Alzheimer's disease and related dementias (“AD/ADRD”). The software is embodied in a digital health application (the “Application”) that is aimed to reduce cognitive and behavioral challenges, reduce isolation and loneliness, and to promote healthy aging. The Application collects data including emotional, physical, and cognitive indicators with each use. Indicators include, but are not limited to, verbal and physical responsiveness, speech complexity and pace, mood, and memory recall. These indicators are biometric analogs for direct clinical observations and tests such as presence of disease, and changes in chronic disease. A similar, near-daily observation cadence of such indicators by clinicians is impractical, time-consuming, and costly. Thus, the data collected over time by the Application provides a basis for long-term trend analysis and detection of changes that precede an acute health event. This alleviates healthcare professional and caregiver responsibilities and can be used for further research and support for PLWD.

According to an embodiment, the Application collects specific medical biometric data analogs that, taken together, indicate the long-term progression of both dementia and chronic diseases. Machine learning then plots the inflection points that can support calculations preceding acute medical events such as UTIs or cardiac, pulmonary, and renal issues.

The Application passively collects a broad set of data with a cadence of, for example, several seconds or less while the PLWD is engaged with the Application. According to an embodiment, the collected data includes, but is not limited to:
a. Emotional: Using emotion recognition software, the Application identifies moment-to-moment changes in emotional states through facial-emotion-recognition and speech (or sounds, if the PLWD is non-verbal) detection. The Application also detects mood changes over time.
b. Cognitive: The Application captures moment-to-moment data comprising the PLWD's responsiveness to engagement stimuli in the interactive virtual world of the Application. In some embodiments, the engagement stimuli comprise queries, prompts, or guidance from an avatar. The PLWD may respond to the stimuli with speech or by touching or tapping specific objects in the virtual world. For speech responses, the Application captures the number of words used, phrasing, speech patterns, pace, and repetition. These and other indicators are used to gauge changes in cognition over time and to detect the rate of change.
c. Physical: Interaction with the Application occurs through verbal and non-verbal communication and by physically tapping a touchscreen of the client device. In an embodiment, the Application detects changes in fine motor skills and hand-eye coordination. The Application calculates the PLWD's reach, precision hitting targets (which may be indicative of tremors), and the speed of execution. Unique repetitive combinations of the above data streams identify, for example, when a person is in pain-even though they may be unable to identify or describe it.

The Application generates a risk profile which provides a clinician the opportunity to observe a risk-profiled patient more closely. The clinician may triage the patient while they are in familiar surroundings, thereby hastening treatment before ED and hospital visits become the only option. According to an embodiment, the risk profile is generated daily but could be generated at any customizable time interval (e.g., hourly, weekly, monthly, etc.).

The Application comprises at least one artificial intelligence (“AI”)-driven avatar existing in a virtual world filled with therapeutic stimuli. In an embodiment, the avatar may comprise a personalized AI companion providing support to PLWD and alleviating caregiver responsibilities.

The avatar may apply an AI-driven dialogue system specific to dementia care to interact with users via empathetic, personalized, and engaging conversations. The dialogue system may be based on an unbiased and representative dataset of the wide spectrum of dementia patients, including a range of ages, races, and severity of dementia, as well as data samples in multiple languages. In an embodiment, in addition to being available in the PLWD's preferred language, the Application comprises culturally meaningful content to increase engagement and ensure ongoing interest. In an embodiment, the dialogue system may be based at least in part on de-identified patient stories or life profiles. Incorporation of patients' narratives encompassing patients' values, religious beliefs, family background, career, hobbies, and the like provides a richer context and more effective conversation topics for the dialogue system.

In an embodiment, the dialogue system may reproduce what a well-skilled dementia caregiver would say. A number of dementia symptoms, such as confusion, memory loss, repeating questions, using unusual words to refer to familiar objects, difficulty speaking, and difficulty understanding and expressing thoughts make conversation more difficult. Therefore, speaking to PLWD effectively requires use of unique conversational strategies. The claimed dialogue system may utilize strategies to improve communication with PLWD, including reducing the number of words used in a sentence, reducing the complexity of sentences by using a maximum of one verb per sentence, keeping questions direct rather than open-ended, providing users with clear choices throughout the conversation, and keeping the conversation at a pace appropriate for each user's abilities in order to give the user time to process what has been said.

With reference to FIG. 1, components of the digital health application are disclosed. According to an embodiment, the Application comprises a multi-agent dementia dialogue system 100. The multi-agent system 100 is configurable to allow domain experts to adjust the Agents (102-118) based on preference and need. The disclosed dialogue system 100 interacts with a PLWD (or the “player”) 101 using real time dialogue. The dialogue is formulated using a multi-modal or mixed model technique which utilizes data modalities such as audio, visual, and touch. This helps better understand the player's 101 needs and condition, ranging from early to late stages of dementia.

In an embodiment, the dialogue system 100 combines rule-based AI with statistical machine learning (“ML”) to create a novel experience for the PLWD 101 for each companion game 122 session of the Application. The Application performs in-depth historical analysis across different data modalities to predict potential emergency situations or to raise concerns regarding specific areas of cognitive decline. The Application monitors each session of gameplay and may suggest areas of focus or changes to the player's 101 approach. Data on the player's 101 progress, communications, skill, and time spent is logged automatically within the Application, while AI-supported feedback provides support, tips, and advice based on evaluated progress data.

According to an embodiment, the companion game 122 of the Application acts as the sensors for the Agents (102-118) by collecting incoming audio, visual, and touch (e.g., tapping) data from the player 101. The companion game 122 provides on-demand social and emotional support through empathetic digital personas (or avatars) in a virtual environment such as a virtual home world. The avatars are AI-driven. The avatars are configured to connect and converse directly with the player 101 to offer conversation, activities of daily living (“ADL”) support, and player-centered activities. Multi-modal emotion recognition and AI-driven personalization allows for optimized user engagement while reducing a caregiver's workload. Increased interaction between the player 101 and the companion game 122 and avatar(s) creates better engagement, allowing the system 100 to fine-tune the health biometric analog data collected and stored in the environment database 120.

According to an embodiment, the environment database 120 comprises a centralized, real time structured query language (“SQL”) database. Agents 102-118 are configured to interface with and update the environment database 120; the Agents 102-118 have access to the environment database 120 and can collect/gather, provide, and update data through their actions. In an embodiment, the environment database 120 is configured to store and organize the most useful and minimal information required to enable the Agents 102-118 to make accurate decisions and to take action leading to an engaging experience for the player 101.

According to an embodiment, the environment database 120 comprises medical data (e.g., age, gender, comorbidities), dementia-specific data, background information, and session data and reports. Dementia-specific data may include dementia severity level (which may be quantified according to a clinical scale), the player's 101 speech capabilities (e.g., talkative, not talkative), and other factors related to dementia which may vary from player to player. Background information may comprise the player's 101 interests, favorite kinds of music/art, family history, religious views, etc. and may be used to create more interesting and relevant dialogue for the player 101. Session data and reports may include facial emotion recognition predictions, speech emotion recognition predictions, speech to text translations, tapping locations, language preferences, object IDs for objects which were tapped, context regarding activities available in the current state of the companion game 122, chat history for the current session, previous and new reports generated for the player 101, and other information which is used in real time to help guide the conversation with the player 101. According to another embodiment, the environment database 120 is a document database but is flexible in design and relationships with components of system 100.

The external databases 126 may contain data or references to data which is added to, but not always kept in, the environment database 120 during a session. The external databases 126 comprise Electronic Health Record (“EHR”) databases or other such relevant databases. According to an embodiment, the external databases 126 are not always part of the AI environment of the system 100. For example, external databases 126 may store an image file while the environment database 120 stores a reference URL to the image. In another example, profile information for a player 101 such as birthday and medical information stored in the external databases 126 may be used during a session or to generate a report but may not always be kept in the environment database 120.

Agents (A1-A9) 102-118 will now be described. Agent (A1) 102 comprises a speech emotion recognition (“SER”) agent configured to process audio signals to make predictions about the PLWD's 101 emotions. Some PLWDs 101 may struggle with forming coherent sentences and may instead mumble, grunt, or moan. However, even if they lack words, these sounds can carry different emotional tones. As such, Agent 102's ability to predict emotion based on audio signals (rather than solely relying on words and images) using, for example, sentiment analysis and Facial Emotion Recognition (“FER”) is incredibly useful. Prediction data generated by Agent 102 is stored as probabilities in the environment database 120. The data is analyzed in either real time (e.g., during dialogue) or over long running processes (e.g., during clinical report creation).

Agent (A2) 104 comprises a reward function agent configured to monitor a player's 101 session in real time. Agent 104 is further configured to compute the system's 100 overall reward based on text-action pairs generated by the coordinated efforts of the other Agents (102, 106-118). For example, a reward may be given when the system 100 generates a response for the player 101 to tap on an item (text), and the player 101 does tap on the item shortly thereafter (action). The functionality of Agent 104 is based on the classical AI theory of Cooperative Multi Agent Systems in which agents who act on their own coordinate with other agents towards the overall system objective. According to an embodiment, the multi-agent dialogue system 100 produces two main outputs—the text to be spoken back to the player 101, as well as any accompanying action data. Action data may include, for example, an identification of an object to be highlighted in the room.

Agent (A3) 106 comprises a clinical report manager (“CRM”) configured to analyze and summarize session data into useful, human-readable reports. The reports may comprise progress data of the player for a given session or over a given number of sessions. Agent 106 is further configured to raise concerns in real time based on insights derived from the report generation process. Specifically, Agent 106 raises concerns about player 101 behavior that it categorizes as abnormal based on historical trends showing states of gradual or rapid decline. Depending on the severity of the concern, this may lead to new treatment plans or medical emergency interventions.

According to an embodiment, Agent 106 generates reports asynchronously and does not influence the dialogue itself. However, if an emergency is predicted based on the incoming data, Agent 106 is configured to send a notification to a real-life caregiver so they may take appropriate action. The notification may comprise, for example, an audio/visual alarm or alert.

Agent (A4) 108 comprises a medical assistant agent configured to collect and organize information from the environment database 120 in real time. This provides Agent 110 the required context to respond to the player 101. Agent 108 utilizes different rules and conditions to compose a prompt based on data such as chat history, background information of player 101, and other relevant information from real time dialogues. The output of Agent 108 is collected by Agent 116, which in turn passes it off to Agent 110 who will ultimately decide what to say to player 101.

Agent (A5) 110 comprises a caregiver agent configured to generate engaging and empathetic responses in real time. According to an embodiment, Agent 110 utilizes a large language model (“LLM”) to generate the responses based on context provided by Agent 108. Agent 110 dynamically adjusts its conversation style based on the provided context regarding the player 101. For example, speech capability may vary drastically from one player to another. As such, different types of conversational techniques which are either more open-ended or more direct may be needed to better suit the individual player 101. The LLM utilized by Agent 110 may be guided to create empathetic responses through prompt engineering, fine tuning, or a combination of both.

Agent (A6) 112 comprises a speech to text (“STT”) agent configured to translate speech audio into text in real time. The translated text is added and stored in the environment database 120. Agent 112 plays an integral role for players 101 who have higher speech capabilities and enjoy conversation. The sessions of these players 101 are more conversational compared to those of players 101 who may struggle with speech.

Agent (A7) 114 comprises a dementia expert agent configured to use the information provided by Agent 108 to decide which action to take based on the category or state of the session from the provided context. According to an embodiment, system 100 utilizes classical AI methods such as a rule-based system, finite state machine, behavior tree, or other generative and statistical models to guide the conversation with the player 101. Not all text responses are the same, and each text response can be categorized into different types of actions. For example, one action could be “continue dialogue”—this action may be triggered when the player 101 has just engaged in conversation with Agent 114. In order to continue the conversation, system 100 would need to create a relevant response to the player's 101 dialogue. As another example, if the player 101 is displaying negative emotion facial expressions, and/or their tone of voice is negative, Agent 114 can decide to take action to “switch topics.”

According to an embodiment, Agent 114 can decide that it does not need Agent 110 to generate a response in order to reduce latency and cost of the system 100. For example, when suggesting to player 101 to tap on an object, Agent 114 may prefer to randomly select from dialogue options such as “Can you please tap on the x?”, “Can you please try tapping on the x?”, or “Hmm, I wonder what this is? Let's try tapping on it.” In an embodiment, Agent 114 may rely on smaller lightweight language models in these narrow dialogue situations as determined on a case-by-case basis.

Agent (A8) 116 comprises a dialogue coordinator agent configured to orchestrate the other Agents (102-114, 118) to create the real time dialogue and return the final output of text-action pairs used in the companion game 122. According to an embodiment, in order to generate a response for the player 101, Agent 116 may: (1) get the translated text from Agent 112 and send it to Agent 108; (2) get the report from Agent 108 and send it to Agent 114; (3) get the decision from Agent 114 and send (or not send) it to Agent 110; (4) in the case where a job was sent to Agent 110, Agent 116 may collect the response from Agent 110 and return it to the companion game 122; and/or (5) if a job was not sent to Agent 110, Agent 116 may return the predetermined response from Agent 110 to the companion game 122.

The real time dialogue module 124 interfaces with Agent 116. The companion game 122 sends session data comprising speech/audio, tapping, and facial expressions to databases 126 and in turn to the environment database 120. The companion game 122 also sends a dialogue job to the real time dialogue module 124 which in turn sends the job to Agent 116. Agent 116 utilizes the data stored in the environment database 120 including session data to create a dialogue response comprising text and/or an action prompt. The dialogue response is conveyed to the player 101 via companion game 122.

According to an embodiment, a dialogue may be coordinated in multiple ways based on varying degrees of reliance on output from Agent 116. For example, in a fully event-driven system, the Agents (102-114, 118) individually monitor the environment database 120 and therefore may not need to relay information to Agent 116. Rather than waiting on a job to be assigned to them, the Agents (102-114, 118) may initiate jobs on their own. Considering preference of utilizing Agent 116 as a coordinator/orchestrator of the other Agents (102-114, 118) vs. having the Agents (102-114, 118) coordinate themselves creates system tradeoffs such as system latency.

Agent (A9) 118 comprises a facial emotion recognition agent configured to predict the player's 101 emotional state based on data, such as incoming images, gathered during the session. As players 101 may struggle with speech to the point of being nonverbal, Agent 118 is a critical component in creating engaging dialogue for the PLWD 101. An effective understanding of the player's 101 needs and interest can heavily depend on the facial expressions of the player 101.

Agent 118 updates the environment database 120 with predicted probabilities associated with each emotion class. According to an embodiment, Agent 118 utilizes deep learning computer vision models to find the face of the player 101 in the video frame and predict the player's 101 emotion according to a two-step process. Agent 118 monitors an image database comprising video frames from a given session, makes predictions based on the incoming images, and updates the environment database 120 with the predictions. According to an embodiment, FER occurs asynchronously from the various audio processes of system 100.

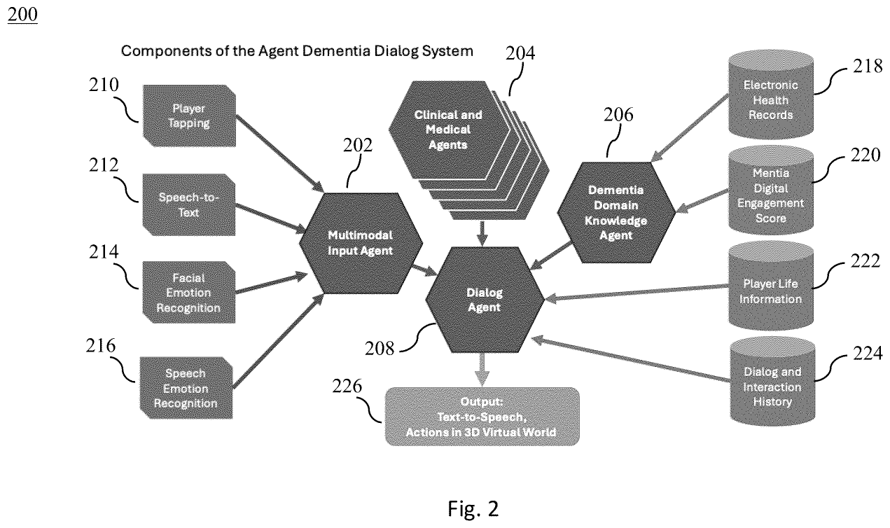

With reference to FIG. 2, components of the digital health application are disclosed. According to an embodiment, the Application comprises an agent dementia dialogue system 200. The architecture of system 200 comprises multiple agents, which may utilize the power of AI to complete their tasks. According to an embodiment, each agent is a piece of code specialized on a given task and configured to cooperate with other agents to generate output.

System 200 records information that is captured during a session in real time and sends it to a multimodal input agent 202. The information includes player tapping 210, speech to text translation 212, FER 214, and SER 216, as well as dialogue, video, and any other session data.

System 200 is configured to interface with external databases including an electronic health records database 218, a digital engagement score database 220, a player life information database 222, and a dialogue and interaction history database 224 to integrate relevant health data.

The EHR database 218 and digital engagement score database 220 interface with a dementia domain knowledge agent 206. The player life information database 222 and dialogue and interaction history database 224 interface with a dialogue agent 208. The multimodal input agent 202, dementia domain knowledge agent 206, and clinical and medical agents 204 provide output to the dialogue agent 208. The dialogue agent 208 produces output 226 comprising text-to-speech dialogue and actions to be performed by a player within the virtual world of the Application.

According to an embodiment, clinical and medical agents 204 comprise a clinical report manager (“CRM”) configured to analyze and summarize session data into useful, human-readable reports. The CRM is further configured to raise concerns in real time based on insights derived from the report generation process. Specifically, the CRM monitors the collected datasets in real time and triggers an alert when such data shows a potential acute health event. When the alert is triggered, the CRM sends information regarding the alert to appropriate medical staff according to preset protocol. For example, an alert may be triggered when system 200 predicts an emergency visit. An alert may also be triggered based on observational statistics showing outliers in the session data (e.g., an unusually high number of negative FER predictions). The reports generated by the CRM include observation statistical summaries which can be viewed during or after or a given session.

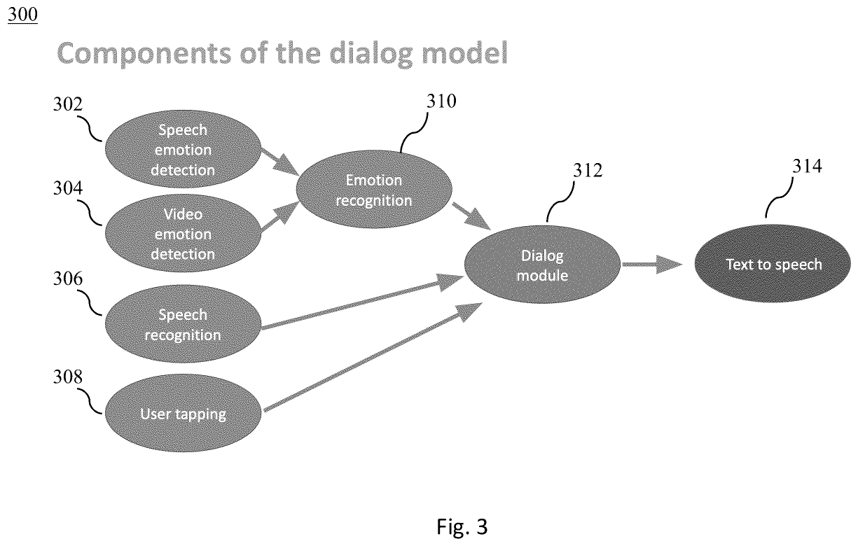

With reference to FIG. 3, components of the dialogue model 300 are disclosed according to an embodiment. Components include emotion recognition 310, which comprises both speech emotion detection 302 and video emotion detection 304, speech recognition 306, and user tapping 308. For example, speech emotion detection 302 may comprise detecting positive, neutral, and negative emotions based on a user's speech, while video emotion detection 304 may comprise detecting positive, neutral, and negative emotions based on a user's facial expression.

Data collected during a session including emotion recognition 310, speech recognition 306 (i.e., speech to text translation), and user tapping 308 are sent to the dialogue module 312. The dialogue module 312 analyzes the gathered data to produce output in the form of text to speech 314 responses-dialogue which is presented to the player during a session.

Text to speech responses 314, or speech synthesis output, are generated from a hybrid of a multi-lingual dialogue dataset according to rule-based algorithms and text created by generative AI with a Large Language Model (“LLM”). Different emotional predictions may output different dialogue reactions from the avatar. For example, the avatar may be configured to output multiple different sentences in response to the same user input depending on the emotional prediction, just as a caregiver would. In another embodiment, responses may be generated according to statistical or data analytic models for dialogue management.

The Application comprises various operating modes. An exemplary operating mode is configured for autonomous use by the PLWD and comprises a plurality of virtual environments and an AI caregiver. Another exemplary operating mode is configured for joint play by a PLWD and their live caregiver. This operating mode provides an opportunity to learn by doing, wherein the AI monitors both the caregiver and the PLWD, offers guidance on how and what to communicate, and suggests activities. Another exemplary operating mode is configured for use by a caregiver as a training mechanism using an AI patient. In all operating modes, the Application utilizes speech recognition, facial recognition, speech analysis, and other tools to determine the user's (i.e., the PLWD and/or their caregiver) mood and respond accordingly.

In an embodiment, the hybrid rule-based-AI dialogue model may be used when the goal is to train a caregiver in dementia management. Using this model, a dementia caregiver can converse and interact with an AI-enabled avatar. The AI-enabled avatar embodies persons with various forms of dementia and various personalities in learning exercises throughout training for better personalized caregiving. For example, a “read/view followed by quiz” approach may be used. The AI system may monitor each session of gameplay and suggest areas of focus or changes to the user's approach. Core competencies may be learned non-linearly by following a loose narrative with prompts, choices, and implications of choice. Data on the user's progress, communications, skill, and time spent is logged automatically within the application, while AI-supported feedback provides support, tips, and advice. In an embodiment, financial incentives may be provided based on achievements, and a course certificate or other prize may be awarded upon completion of gameplay. All such incentives may be available to both users with dementia and caregivers.

For example, the Application may provide a prompt about modesty, stating, “Inside the bathroom, the host, Robin, is ready to shave. You are shy to see a man shave in front of you. How will you handle it?” In this example, the caregiver can practice conversations using a prompt/respond/observe approach.

In another embodiment, the hybrid data analytic dialogue model may be used by dementia caregivers to perform sessions with a PLWD. The caregiver may be able to determine the level of automation of the given dialogue model. The text responses may be converted to speech in the form of audio and video output via the application. Thus, the caregiver may practice communicating and sharpen their communication skills all while engaging with the learn-by-play training system of the present invention.

The one or more avatars may be female (e.g., “Julie”) or male (e.g., “Robin”). The avatar may act as a host who is available to welcome and help the user but may ask the user for help as well. Caregivers may also participate by playing with the Application along with the PLWD. When played together, the Application trains both formal and informal caregivers in dementia management-a crucial aspect of home care-through “learning by doing” methods. Using the dialogue mode, the Application observes how and what the caregiver is communicating, observes how the PLWD is reacting and provides suggestions to the caregiver as needed to improve interaction with the PLWD. Such suggestions may include, for example, a change in tone or words used, or to proceed to other activities known to improve the PLWD's mood.

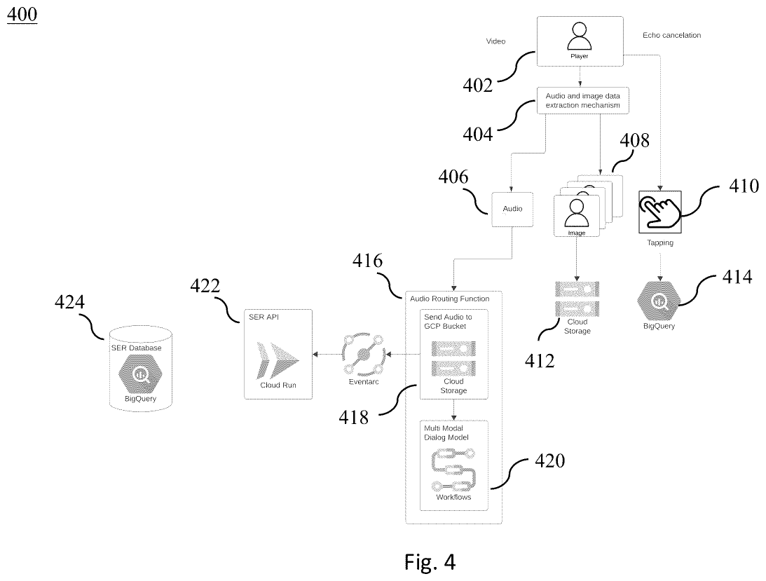

With reference to FIG. 4, a flow diagram of an integrated model architecture 400 of the digital health application is disclosed according to an embodiment. One or more of Natural Language Processing and LLMs are combined with a multi-modal or mixed model technique wherein users' speech patterns, facial expressions, screen tapping patterns, and other input are assessed to gather information about the users and to respond accordingly. Audio data 406 and image data 408 are extracted from video input from the user (or “player”) 402 via an extraction mechanism 404. In an embodiment, echo cancellation technology may be applied to the video input to ensure clarity of the audio. Once extracted, the image data is stored in cloud storage 412. Extracted audio data undergoes an audio routing function 416, wherein the audio data is stored in cloud storage 418 and used in multi-modal dialogue model workflows 420. The output of the audio routing function 416 is sent to a speech emotion recognition application programming interface (“API”) 422. Speech emotion that has been extracted from user audio is stored in a speech emotion recognition database 424. The user's 402 screen tapping patterns 410 are also assessed and collected in a database 414.

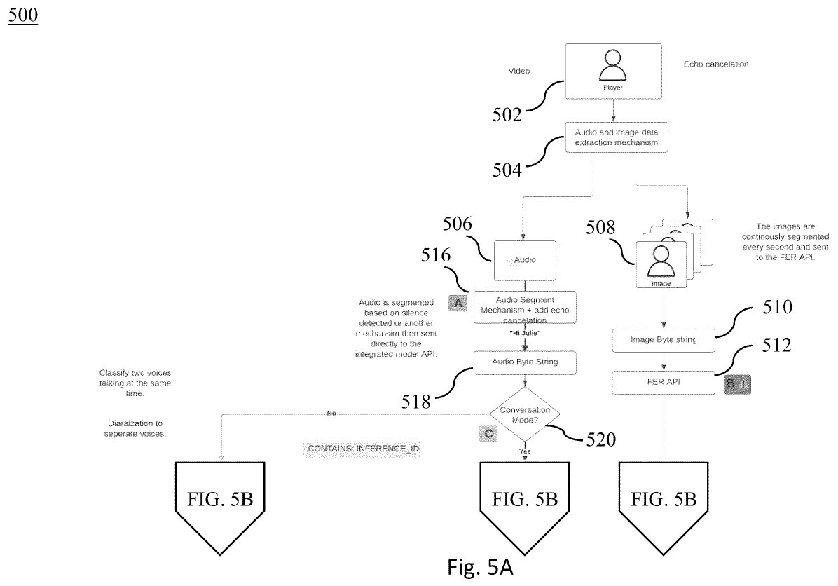

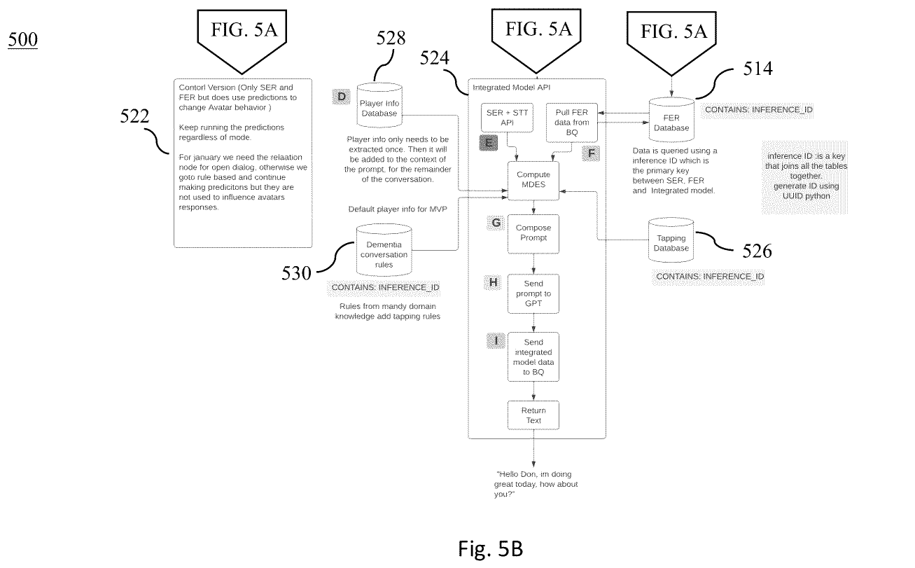

With reference to FIGS. 5A-5B, a flow diagram of an integrated model architecture 500 of the digital health application is disclosed according to an embodiment. Audio data 506 and image data 508 are extracted from video input from the user 502 via an extraction mechanism 504. Once extracted, the images from the video input are continuously segmented every second using an image segment mechanism. The images are then sent as an image byte string 510 (e.g., in Python) to a facial emotion recognition API 512. The image data is then stored in a facial emotion recognition database 514. The user's screen tapping patterns are also assessed and collected in a tapping database 526.

Information about the user is extracted and stored in a player information database 528. This information only needs to be extracted once, after which it is added to the context of the prompt for the remainder of the avatar-driven conversation. Rules from domain knowledge regarding conversations with PLWD are stored in a dementia conversation rules database 530.

Extracted audio is segmented using an audio segment mechanism 516, for example, based on silence detected or based on another mechanism. Echo cancellation technology may be applied to the audio stream to ensure clarity of the audio. Speaker diarization may also be applied to the audio stream to separate voices and group together speech segments on the basis of speaker characteristics. The model determines whether the Application is operating in conversation mode 520. If the Application is operating in conversation mode, the audio is sent to an integrated model API 524 as an audio byte string 518 (e.g., in Python). If it is not in conversation mode, only speech emotion recognition and facial emotion recognition are utilized while predictions determine the behavior of at least one avatar 522.

Within the integrated model API 524, the speech emotion recognition and speech to text API, facial emotion recognition data from the facial emotion recognition database 514, tapping pattern data from the tapping database 526, player information from the player information database 528, and dementia conversation rules from the dementia conversation rules database 530 are used to compute a Mentia Digital Engagement Classification (“MDEC”). Based on the computed MDEC and detected speech, a prompt is composed and sent to an LLM, and text is returned. As a result, when the user engages with the Application by speaking (e.g., “Hi Julie”), the integrated model API 524 causes the avatar to respond (e.g., “Hello, Don, I'm doing great today. How about you?”).

Data in the facial emotion recognition database 514 and the tapping database 526 is queried using an inference ID, which is the primary key between speech emotion recognition, facial emotion recognition, and the integrated model. The player information database 528, as well as the dementia conversation rules database 530, may also be queried using an inference ID. The inference ID may be generated using a Universally Unique Identifier (“UUID”) (e.g., in Python).

The avatar may interact with a user to actively guide them through ADLs, such as personal hygiene tasks, and to deliver therapies. The avatar is configured to deliver pre-determined, AI-based dialogue wherein responses are triggered by input from a user. For example, the avatar may thank or congratulate the user when some action is performed, or a task is completed. In an embodiment, if there is no user input for a longer than usual period of time for a particular user, the avatar delivers a diversional, repetitive, or related prompt based on the MDEC and emotion parameters, aimed at creating a response from the user to ensure continued engagement.

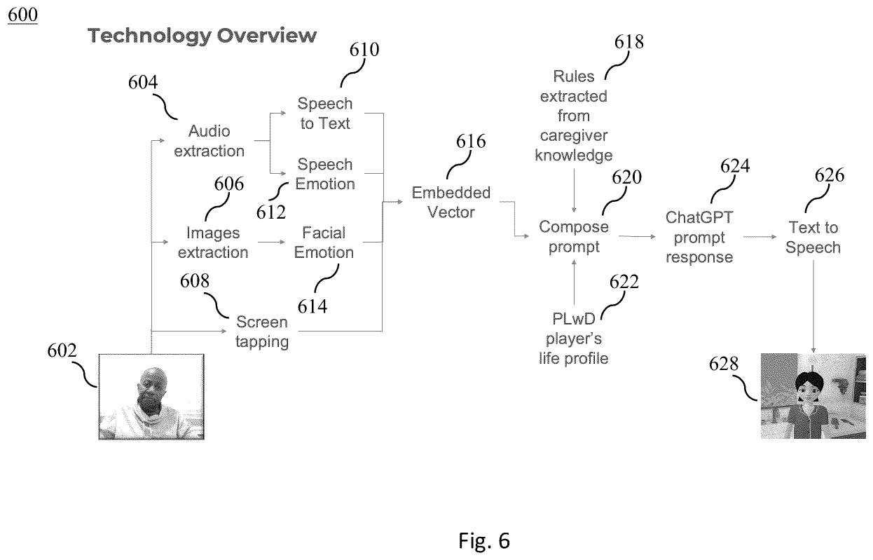

With reference to FIG. 6, a technology overview flow diagram 600 of the digital health application is disclosed according to an embodiment. Audio data 604 and image data 606 are extracted from a user's video input 602. Speech emotion 612 is extracted from the audio data and facial emotion 614 is extracted from the image data. The audio data also undergoes speech to text synthesis 610. Screen tapping input 608 is also collected from the user. Collectively, this data is analyzed and combined into an embedded vector 616 which is used to compose a prompt 620. The user's life profile 622 as well as rules extracted from caregiver knowledge and a computed MDEC 618 are also utilized in the composition of the prompt 620. With the support of an LLM, a prompt response is generated 624 and undergoes text to speech synthesis 626 to be delivered to the user via the avatar 628.

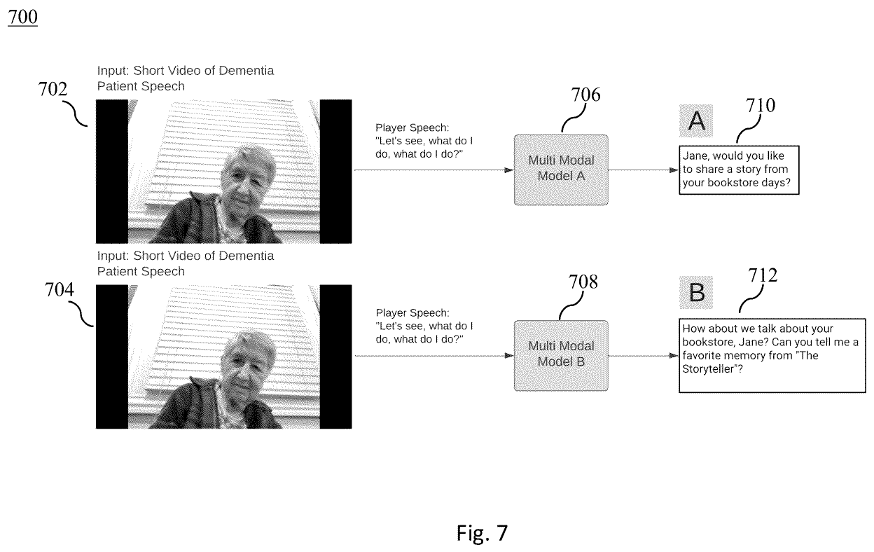

With reference to FIG. 7, a flow diagram of a user interacting with an integrated model architecture 700 of the digital health application is disclosed according to an embodiment. Video input in the form of user speech may result in a different prompt depending on which multi-modal model is used. For example, when the user says, “Let's see, what do I do, what do I do?” (input 702, 704), the avatar may deliver a prompt such as, “Jane, would you like to share a story from your bookstore days?” (output 710), when configured to a given multi-modal model 706. When configured to a second given multi-modal model 708, the avatar may deliver a prompt such as, “How about we talk about your bookstore, Jane? Can you tell me a favorite memory from ‘The Storyteller’?” (output 712).

The dementia-friendly interface delivers individualized, person-centric digital content meaningful to each PLWD. It uses the universal language of animation, engagement stimuli, and interaction design to deepen understanding of a patient's individuality. The focus of the Application is on the autonomy of PLWD, with a goal of helping PLWD to accomplish activities of daily living. In an embodiment, an acute health risk profile is created for each user based on user input throughout gameplay. Thus, the Application may be configured to identify and flag a user's physiological issues.

The Application may comprise scenario-based guided activities for users to complete. These may comprise specific dementia care and guided personalized communication goals developed based on peer-reviewed dementia care and human computer interaction literature. An exemplary activity may comprise a guided goal of preparing for bed, either by the user themselves during autonomous play or guided play alongside a caregiver, or by the caregiver during training. Completing a quest positively impacts a user's score and results in a reward in the form of experience points. Completing a quest allows users access to the next environment to embark on one or more new quests. In an embodiment, occasional power-up opportunities to earn more experience points by diving deeper or more frequently into an activity may be provided.

In an embodiment, the Application is built using a 3D game engine. The Application may be accessible on a client device. In an embodiment, client devices may comprise computing devices and may vary in terms of capabilities or features, for example, a cell phone, smart phone, tablet computer, laptop, television, and in-dash car computer, or the like. The client device may be web-enabled and may include one or more physical or virtual keyboards, mass storage, a webcam, a speaker, one or more accelerometers, one or more gyroscopes, global positioning system (“GPS”) or other location identifying type capability, and a display with a high degree of functionality, such as a touch-sensitive color 2D or 3D display.

The Application comprises at least three virtual interactive environments, such as a living room, a bedroom, and a garden. The Application can also include any other 3D room including but not limited to a barn paddock and pasture, a bathroom, a café, a temple, or a zoo. The environments may also comprise a virtual barn, paddock, and pasture to deliver equine therapy, and a bathroom to engage with the top ADLs that support health maintenance. In addition to the at least one avatar, the Application may also comprise pets and farm animals. The claimed Application immerses users in an animated 3D graphical world, creating visceral experiences, cognitive scaffolding, and just-in-time, actionable feedback. The Application may also comprise additional environments that evoke sensory responses, the Montessori approach to activity design, the avatar's use of validation techniques, and enrichment activities that are inspired by real-world evidence-based non-pharmacological treatments.

In addition to the avatar dialogue, the Application may play music and other sounds or tones during gameplay. For example, farm animals may make various animal noises and completion of an activity may result in a triumphant tone or song. The Application may also include theme-related sensory objects, such as perfume in the bathroom, chocolates in the living room, hay in the barn, and fruit in the garden, which users are able to interact with during gameplay.

In an embodiment, the Application comprises at least one anchor, i.e., a live mentor assigned to a user and troubleshoots as needed. Each anchor is experienced in dementia care and is a specialist in the claimed Application. In addition to acting as a resource for those needing emotional and practical support, anchors also receive real time reports via a dedicated dashboard to assess skills acquired, competence, change in attitudes, and the like from their assigned user(s).

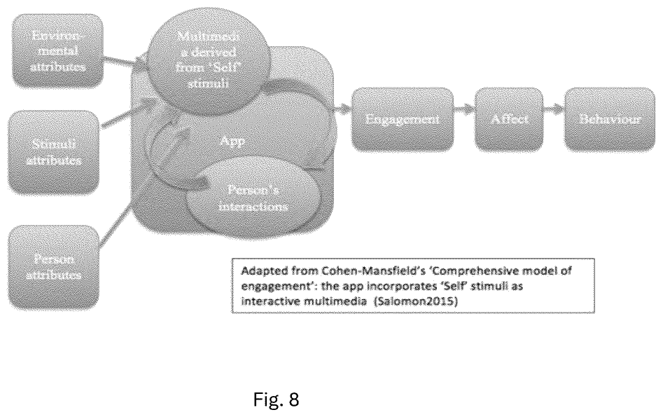

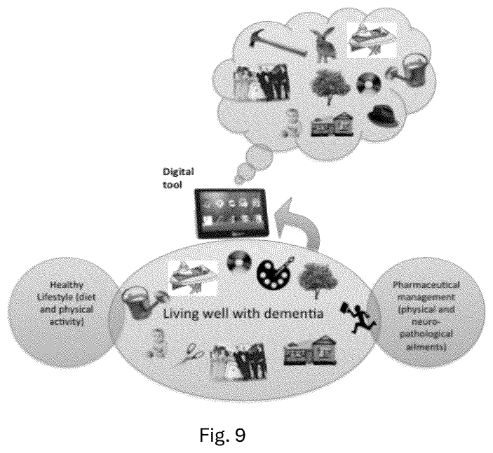

With reference to FIG. 8, in some embodiments, an adapted model of digital engagement is applied wherein the Application is both the environment stimuli interaction and the personal stimuli interaction. The technology can be visualized as a filter through which stimuli flow. In some embodiments, the Application provides a link between dementia's virtuality and virtual worlds, and this, along with a deepening understanding of people with dementia's needs, drives the Application. In some embodiments, the Application is based on the premise that engagement is a vital factor in the effective delivery of activity-based interventions for people with dementia and that engagement can be achieved when the Application meaningfully situates self-stimuli. Sabat's formulation of Selves: 1, 2 and 3 and research into the underlying meaning of stimuli have informed the developing concept for Applying Virtual Environments for Dementia Care (“AVED”) worlds, which is a construction of settings (for situated-ness), scenarios (contextual events) and interactional objects (opportunities for agency). This is demonstrated by a diagram as seen in FIG. 9.

In some embodiments, ten design principles govern the implementation of digital engagement and govern the Application's construction. These ten design principles are not fixed in stone. As grounded theory demands, these are modifiable as new implications from the research that emerges. Refinements can be made as a result of consultations with members of the dementia community. Principle 1 maintains that a virtual world is a resource and stimulator for multiple constructions of Self (the Sabat Model). To make the narratives coherent, interaction inputs and timings are be calibrated to suit the abilities of users. Content is extensible and modifiable to engender a personalized experience. In some embodiments, the Application is installed on a portable tablet device, such as an iPad or Galaxy, allowing the Application to go wherever the players are, which adds to the sense of the extended self.

Principle 2 involves pairing dementia engagement strategies with those of virtual worlds. In a virtual world, one's intention is enacted using an embodied command (e.g. tapping on the tablet screen). Simultaneously, a different action occurs within the virtual world (e.g. the car engine starts). The two engagement areas don't need to be separate; they can be integrated. In Table A below, well-established actual world engagement attributes are matched to engagement attributes of virtual worlds. The Application's settings, interactional objects, and streaming media are appropriately selected and configured, allowing users to respond with the same sensitivity to its engagement stimuli as they do with the engagement stimuli they encounter in the actual world.

TABLE A
Engagement ledger: virtual worlds and person-centered care

| Engagement attributes of virtual | |
|---|---|
| worlds (VW) | Engagement attributes of person-centered care |
| VW host multiple activities within | Target the activity to the person, and be cognizant of |
| the world such as games, puzzles, | what is meaningful to them. |
| role play, reading, and media | |
| consumption. | |
| A VW system comprises settings, | Engagement stimuli should be readily available. |
| scenarios and interactional objects. | Engagement Stimuli might include a wallet, wood |
| | puzzle, a baby doll, a respite video, flower arrangement. |
| VWs host multiple players either on | Activities should have “a socialization schedule so that |
| the one device (co-located), | residents receive one-on-one interaction”. Caregivers |
| distributed (online). | (formal and informal) are part of the therapeutic |
| | partnership and benefit from positive outcomes. |
| Programmed objects behave in | Consistent routines engender a sense of security and |
| coherent ways; mechanisms for | are enabling for the person and caregiver. Disrupted |
| controlling the environment are | routines have a negative impact. |
| consistent. | |
| The environment is persistent | Resident's choices should be respected and, as far as |
| (changes made by the user remain | possible, implemented. Agency is empowering. |
| intact) | |
| Streaming multimedia (e.g. music, | Recognition of culturally and linguistically diverse |
| audio, film, images) is hyperlinked | backgrounds; Culturally specific content should be |
| or archived within the app. | accessible. |
| Physics, like shadows, the wind, | Contact with the natural world is a human right; it |
| sounds and other natural elements. | improves mood and well-being. |
| Embodied interaction (agency); | Person-centered care entails the person receiving care |
| Embodied presence (avatars and | having agency; Social interaction is a human need. |
| agents). | Positive social positioning supports well-being. |
| Engagement attributes of virtual | Engagement attributes of person-centered care |
| worlds (VW) | |
| An environment that users can build | Home is a physical and metaphysical concept which |
| or modify. | carries with it conceptualizations of wellness, |
| | independence, social participation, autonomy and |
| | control over one's environment. |
| Captures, stores and replays action | It is important for people with dementia to have success |
| and events in the world at the user's | when recalling information; a memory book is one way |
| call. | to engender “relatively effortless acquisition and |
| | retrieval of new information”. |
| Narrative is explored through | “As the person's ability to communicate linguistically |
| visual signposts and symbols; | declines, he/she may resort to other semiotic resources |
| user's journey advances by chatting | using his/her body to physically enact and perform |
| with others and through gesture, | memories of past events, as well as making use of the |
| using a touchscreen or games | conversational partners' semiotic and cognitive |
| console. | resources.” |

Principle 3 involves creating a virtual world home as a setting for in-app scenarios and interactional objects. The concept of home is framed as a socio-cultural construction within which cultural beliefs and social diversity come into play. The concept of home evokes a sense of well-being, one that is pleasant, light and open, and evokes warmth. It should suggest “healthy living”, which has been described as a sense of having the resources for an everyday life that is satisfying to oneself and others. In some embodiments, the virtual home symbolizes the metaphysical home, based on commonly held values like peacefulness, safety, and orderliness. Drawing upon the social constructionist sense of Self, the home setting is a mix of visual, aural, and emotional stimuli.

Principle 4 involves the virtual world being “dementia-friendly.” This includes but is not limited to the user setting in the Application and dementia-friendly design decisions within the Application. For example, unfriendly stressors commonly encountered in care homes are long hallways with multiple resident rooms on both sides, prominently positioned nursing stations, a lack of outdoor space and consistent background noise. Social environment stressors are unfamiliar people, crowds, and a lack of communication strategies. A dementia-friendly environment is more than a checklist list of optimal architectural and interior considerations. It is about how people with dementia experience their environment. The government bureau Vic Health has advised that a dementia-friendly design should promote positive activities such as the continuation of personal lifestyles, and should encourage curiosity. They have suggested a range of room sizes: private and cozy spaces for quiet time, and larger open ones to cater for family visits and celebrations. The spaces should engender relationship building between staff and resident. Dedicated areas for cooking, craftwork, or listening to music and so on can enhance this.

Principle 5 involves access to outdoors spaces, for example gardens in the virtual world. Such access promotes mental well-being and a broader sense of belonging. Mental and physical needs can be met by being outside, and person-centered care should be built into the core thinking of outdoor designs, including an awareness of people's histories. In some embodiments, the Application's outdoor regions should inspire through its beauty, growth (using animation) and sounds (bird calls, as an example). Depending on a person's preferences, the setting should evoke relaxation and peaceful contemplation, and be a place for activity, such as planting fruit and vegetables.

Principle 6 involves integrating meaningful stimuli into the Application. For example, in some embodiments, the Application aims to be person-centered by containing activities that encourage individuality, choice, and independence. The Application design strives to preserve dignity and self-respect of players, which is the basis of a positive approach to living with dementia in the actual world.

Principle 7 revolves around a world of reminiscence. Traditionally, reminiscence therapy uses a compendium of ephemera (e.g., magazines, newspapers, brochures and posters), audio, music and film/video from earlier times, as a well as a person's photographs or items from a memory box. Places, such as buildings, gardens and historical locations create a context for people's uploaded personal digital artefacts. Another design concept comes from research into the role emotions play in reminiscence. It has been found that memories associated with emotions are more accessible than memories of procedures and tasks. Emotions and feelings associated with the remembered lived experience can reshape conceptions of Self in more effective ways than rationalizing, which may be a process of what was achieved and what was not. In some embodiments, the content inside the virtual world evokes emotional responses from its users.

Principle 8 involves the concept of a virtual companion. In some embodiments, the Application includes an animated person within a virtual world for the user to interact with. An embodied agent could be a mechanism for satisfying the relational aspect of Self, which includes needing to nurture (parental) and be nurtured (the child). In some embodiments, the Application companion's gender, shape, size, and so on is determined in consultation with intended users.

Principle 9 incorporates the role of significant others of the user within the Application. In some embodiments, the Application design recognizes that close family members and care partners are on a dementia journey too and that there is a correlation between the well-being of the informal caregiver and the care recipient. The Application can be a shared activity between a person with dementia and a caregiver. Through dyadic play in the Application, formal caregivers, family, and friends have another resource at their disposal for fostering empathy and understanding for the patient. There is the prospect that communication can flow, ensuring a more enriched visit than might otherwise be the case.

Principle 10 allows the Application to draw on the experience of participants with dementia as design collaborators. In some embodiments, the design phase is a collaborative one with the prototype being an outcome of the participatory process undertaken.

In some embodiments, the Application includes three content-driven elements: settings (e.g., virtual places), scenarios (e.g., storylines relating to settings), and interactional objects (e.g., actionable components that primary players respond to and to which as a result of their interactions, changes occur, confirming to players their active role in the experience).

In some embodiments, primary players are motivated to explore within the Application because of its embedded Self-stimuli and the role that underlying stimuli play in engagement. For example, engagement is more likely to occur when activities relate to an individuals' work and social roles, and personal attributes.

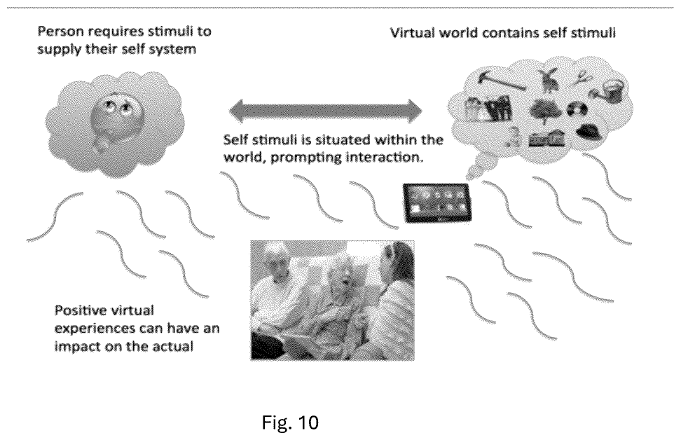

In some embodiments, the Application promotes higher order needs such as belonging, esteem, and self-actualization. In some embodiments, higher order needs are achieved by incorporating engagement traits of well-regarded psychosocial activities into the Application. This process is described diagrammatically, with each diagram being a refinement of the previous one (FIG. 8, FIG. 9, and FIG. 10).

In some embodiments, the Application is a narrative proposal with suggested settings, scenarios, and interactional objects. In some embodiments, the Application includes engagement prompts. For example, engagement prompts are a range of high-quality pictograms depicting people, places, and objects and activities in domestic settings. The engagement prompts promote participation and amplify “voices” without relying too much on words to learn about participant views, attitudes, and responses.

In certain embodiments, “Settings” prompts are based biographic information collected in the participant care profiles. “Settings” prompts can include house exteriors, kitchens, living rooms, and bedrooms. Outdoors settings can include gardens to landscaped parkland and wooded forests.

In certain embodiments, an iPad is used as a platform for the Application and supplies users with ways to input their selections and commands. In certain embodiments, touching, pressing, tapping, drag-and-drop, blowing, swiping, pinching, finger tracing, pushing buttons, and speaking into the built-in microphone are all ways in which the users can interact with the Application. For example, tapping on a fridge in the Application can open the fridge door and further tapping can make food appear and so on. When a participant taps on the virtual companion, the virtual companion can say something to the participant. The intra-world chat encourages and motivates the primary player to continue the activity. In some embodiments, tapping can trigger secondary actions such as objects moving automatically into the correct spot instead of having to be dragged there. These would be pre-loaded so that normal drag-and-drop or other fine motor actions would not be required, and to reduce interactional steps to a minimum.

In some embodiments, the storytelling narrative of the Application can be developed around common social activities (e.g. reminiscence, singing and dancing, arts and craft, flower arranging, and gardening), along with therapeutic interventions such as simulated presence Stimuli in the virtual world are an editable digital code, meaning there are innumerable ways to present them. In some embodiments the Application includes digital building blocks called engagement memes. For example, engagement memes can be tunes, ideas, catch phrases, clothes, ways of making pots, or of building arches, etc. Engagement memes can take any number of forms; the activity need not mimic the actual world version to be present.

In some embodiments, the Application features three interactive 3D graphical settings: the Lounge, the Bedroom, and the Garden. Objects, colors, animations and interactive play have been designed according to the ten design principles (e.g., as described above) formulated from an analysis of existing texts. The Application offers users a range of easy-to-use, interactive 3D experiences presented in a loose, non-linear narrative. In some embodiments, the Application can be explored using a touchscreen tablet device with a modified touchscreen interface (for example the usual zooming and swiping mechanisms can be disabled). Participants have a sense of agency by tapping on screen-based stimuli that interest them and receiving feedback from their actions, both on screen and through the support player's responses. Although the claimed dialogue model specific to dementia care is discussed herein as applied to a digital health application, it may be used in other technologies as well. For example, the dialogue model may be used in a video game, in robots (including humanoid companions such as the Paro robot and more technological robots such as the Neo robot), in interactive avatars on big display screens located in senior communities, in devices such as Alexa, HomePod, Google Hub, or the like, and in commercial customer service applications.

The particular processing operations and other system functionality described in conjunction with the flow diagrams in the Figures are presented by way of illustrative example only and should not be construed as limiting the scope of the disclosure in any way. Alternative embodiments can use other types of processing operations. For example, the ordering of the process steps may be varied in other embodiments, or certain steps may be performed at least in part concurrently with one another rather than serially. Also, one or more of the process steps may be repeated periodically, or multiple instances of the process can be performed in parallel with one another in order to implement the disclosed embodiments.

Functionality such as that described in conjunction with the processes in the Figures may be implemented at least in part in the form of one or more software programs stored in memory and executed by a processor of a processing device such as a computer or server. As will be described herein, a memory or other storage device having executable program code of one or more software programs embodied therein is an example of what is more generally referred to herein as a “processor-readable storage medium.”

FIGS. 1 through 10 are conceptual illustrations allowing for an explanation of the disclosed embodiments of the invention. Notably, the figures and examples above are not meant to limit the scope of the invention to a single embodiment, as other embodiments are possible by way of interchange of some or all of the described or illustrated elements. Moreover, where certain elements of the disclosed embodiments can be partially or fully implemented using known components, only those portions of such known components that are necessary for an understanding of the disclosed embodiments are described, and detailed descriptions of other portions of such known components are omitted so as not to obscure the disclosed embodiments. In the present specification, an embodiment showing a singular component should not necessarily be limited to other embodiments including a plurality of the same component, and vice-versa, unless explicitly stated otherwise herein. Moreover, terms in the specification or claims are not intended to be ascribed an uncommon or special meaning unless explicitly set forth as such. Further, the disclosed embodiments encompass present and future known equivalents to the known components referred to herein by way of illustration.

It should be understood that the various aspects of the embodiments could be implemented in hardware, firmware, software, or combinations thereof. In such embodiments, the various components and/or steps would be implemented in hardware, firmware, and/or software to perform the functions of the disclosed embodiments. That is, the same piece or different pieces of hardware, firmware, or module of software could perform one or more of the illustrated blocks (e.g., components or steps). In software implementations, computer software (e.g., programs or other instructions) and/or data is stored on a machine-readable medium as part of a computer program product and is loaded into a computer system or other device or machine via a removable storage drive, hard drive, or communications interface. Computer programs (also called computer control logic or computer-readable program code) are stored in a main and/or secondary memory, and executed by one or more processors (controllers, or the like) to cause the one or more processors to perform the functions of the invention as described herein. In this document, the terms “machine readable medium,” “computer-readable medium,” “computer program medium,” and “computer usable medium” are used to generally refer to media such as a random access memory (RAM); a read only memory (ROM); a removable storage unit (e.g., a magnetic or optical disc, flash memory device, or the like); a hard disk; or the like.

The foregoing description will so fully reveal the general nature of the disclosed embodiments that others can, by applying knowledge within the skill of the relevant art(s) (including the contents of the documents cited and incorporated by reference herein), readily modify and/or adapt for various applications such specific embodiments, without undue experimentation, without departing from the general concept of the disclosed embodiments. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

### APPENDIX

The following list of references include references containing additional information on Dementia and treatments for PLWD and are hereby incorporated by reference in their entirety.
(1) Alzheimer's Association. (2023). 2023 Alzheimer's Disease Facts and Figures. Alzheimer's & Dementia, 19 (4). https://doi.org/10.1002/alz.13016.
(2) Tahami Monfared, A. A., Byrnes, M. J., White, L. A., & Zhang, Q. (2022). The Humanistic and Economic Burden of Alzheimer's Disease. Neurology and therapy, 11 (2), 525-551. https://doi.org/10.1007/s40120-022-00335-x.
(3) Dewing, J., & Dijk, S. (2016). What is the current state of care for older people with dementia in general hospitals? A literature review. Dementia (London, England), 15 (1), 106-124. https://doi.org/10.1177/1471301213520172.
(4) Shepherd, H., Livingston, G., Chan, J., et al. (2019). Hospitalization rates and predictors in people with dementia: A systematic review and meta-analysis. BMC Medicine, 17, 130. https://doi.org/10.1186/s12916-019-1369-7.
(5) Powell, K. R., Farmer, M., Liu, J., & Alexander, G. L. (2024). A Survey of Technology Abandonment in US Nursing Homes. Journal of the American Medical Directors Association, 25 (1), 6-11. https://doi.org/10.1016/j.jamda.2023.09.002.
(6) Bundy, H., Frazier, L., Woodward, J. M., Liu, T.-L., Taylor, Y. J., Rossman, W., & Mangieri, D. A. (2022). The benefits of virtual in-clinic memory care for rural patients with dementia: Preliminary data. Journal of the American Geriatrics Society, 70 (7), 1874-1876. https://doi.org/10.1111/jgs.17712.
(7) Bass, D. M., Judge, K. S., Maslow, K., Wilson, N. L., Morgan, R. O., McCarthy, C. A., Looman, W. J., Snow, A. L., & Kunik, M. E. (2015). Impact of the care coordination program “Partners in Dementia Care” on veterans' hospital admissions and emergency department visits. Alzheimer's & Dementia: Translational Research & Clinical Interventions, 1 (1), 13-22. https://doi.org/10.1016/j.trci.2015.03.003.
(8) Zwakhalen, S. M. G., Koopmans, R. T. C. M., Geels, P. J. E. M., Berger, M. P. F., & Hamers, J. P. H. (2007). The prevalence of pain in nursing home residents with dementia measured using an observational pain scale. European Journal of Pain, 13, 89-93.
(9) Yourman, L. C., Kent, T. J., Israni, J. S., Ko, K. J., & Lesser, A. (2020). Association of dementia diagnosis with urinary tract infection in the emergency department. Journal of the American College of Emergency Physicians open, 1 (6), 1291-1296. https://doi.org/10.1002/emp2.12268.
(10) Masman, A. D., van Dijk, M., van Rosmalen, J., Baar, F. P. M., Tibboel, D., & Boerlage, A. A. (2018). The Rotterdam Elderly Pain Observation Scale (REPOS) is reliable and valid for non-communicative end-of-life patients. BMC Palliative Care, 17 (1), 34. https://doi.org/10.1186/s12904-018-0280-x.
(11) Fuchs-Lacelle, S. & Hadjistavropoulos, T. (2004). Development and preliminary validation of the pain assessment checklist for seniors with limited ability to communicate (PACSLAC). Pain Management Nursing, 5 (1), 37-49.
(12) Dutta, C., Pasha, K., Paul, S., Abbas, M. S., Nassar, S. T., Tasha, T., Desai, A., Bajgain, A., Ali, A., & Mohammed, L. (2022). Urinary Tract Infection Induced Delirium in Elderly Patients: A Systematic Review. Cureus, 14 (12), e32321. https://doi.org/10.7759/cureus.32321.
(13) Mansour, S., & Nogues, S. (2022). Advantages of and Barriers to Crafting New Technology in Healthcare Organizations: A Qualitative Study in the COVID-19 Context. International journal of environmental research and public health, 19 (16), 9951. https://doi.org/10.3390/ijerph19169951.
(14) Hamblin K. A. (2022). Technology in care systems: Displacing, reshaping, reinstating or degrading roles?. New technology, work and employment, 37 (1), 41-58. https://doi.org/10.1111/ntwe.12229.
(15) Assessing a Novel Virtual Environment That Assists With Activities of Daily Living. ClinicalTrials.gov ID NCT05418296. Sponsor, Mentia DTx. Available at https://clinicaltrials.gov/study/NCT05418296.
(16) Cohen-Mansfield, J., Dakheel-Ali, M., & Marx, M. S. (2009). Engagement in persons with dementia: the concept and its measurement. The American journal of geriatric psychiatry: official journal of the American Association for Geriatric Psychiatry, 17 (4), 299-307. https://doi.org/10.1097/JGP.0b013e31818f3a52.
(17) Gitlin, L. N., Kales, H. C., & Lyketsos, C. G. (2012). Nonpharmacologic management of behavioral symptoms in dementia. JAMA, 308 (19), 2020-2029. https://doi.org/10.1001/jama.2012.36918.
(18) Newsletter December 2023, “Featured JH AITIC Year 1 Awardee: Mentia” https://aitc.jhu.edu/newsletters/.
(19) O'Reilly, K. (2011). Ethnographic Methods (2nd ed.). London: Routledge. https://doi.org/10.4324/9780203864722.
(20) Siegel, D. (2012). Usability for engaged users: The naturalistic approach to evaluation. In J. A. Jacko (Ed.), Human Computer Interaction Handbook (3rd ed., pp. 1243-1247). Boca Raton: CRC Press. http://doi.org/10.1201/b11963-52.
(21) Reisberg, B., Ferris, S. H., de Leon, M. J., and Crook, T. The global deterioration scale for assessment of primary degenerative dementia. American Journal of Psychiatry, 1982, 139:1136-1139.
(22) Salomon, Amanda “Expressing ‘Self’ in a virtual world: Digital engagement for people with moderate to severe dementia” (PhD Thesis 2016) https://researchbank.swinburne.edu.au/items/75d5faf6-16cd-45bd-9e77-bd9f780d38a1/1/.

What is claimed is:

1. A system for digital engagement comprising a processor coupled to memory, the processor being configured to:
operate an application for a patient with dementia, wherein the application comprises at least one applied virtual environment;
evaluate input of the patient based on a mixed model for observing digital engagement;
engage the patient with the application using dialogue delivered by at least one virtual avatar, the virtual avatar being supported by artificial intelligence and the dialogue being determined based on the evaluated input;
log and evaluate progress data of the patient; and
provide feedback and support based on the evaluated progress data.

2. The system of claim 1, the system further comprising a plurality of agents supported by artificial intelligence, wherein each of the plurality of agents is configured to perform a given task and to cooperate with others of the plurality of agents to generate output.

3. The system of claim 1, wherein the input is evaluated using one or more of speech emotion detection, video emotion detection, speech recognition, and user tapping.

4. The system of claim 1, wherein the processor is configured to evaluate the input of the patient and deliver the dialogue in real time.

5. The system of claim 1, wherein the processor is further configured to:
predict a potential emergency based on the evaluated input; and
trigger an alarm based on the prediction.

6. The system of claim 1, wherein the dialogue is formulated based on the evaluated input of the patient.

7. The system of claim 6, wherein the processor is further configured to tailor the dialogue based on the patient's medical data, a severity of the patient's dementia, the patient's speech capability, and background information regarding the patient.

8. The system of claim 1, wherein the processor is further configured to engage the patient with the application using guided personalized goals.

9. The system of claim 1, wherein the processor is further configured to evaluate progress data of the patient including a progression of dementia over a given period of time.

10. The system of claim 1, wherein the applied virtual environment comprises any 3D room including but not limited to a living room, a bedroom, a garden, a barn paddock and pasture, a bathroom, a café, a temple, or a zoo.

11. A method for digital engagement performed by a processor coupled to memory, the method comprising:
operating an application for a patient with dementia, wherein the application comprises at least one applied virtual environment;
evaluating input of the patient based on a mixed model for observing digital engagement;
engaging the patient with the application using dialogue delivered by at least one virtual avatar, the virtual avatar being supported by artificial intelligence and the dialogue being determined based on the evaluated input;
logging and evaluating progress data of the patient; and
providing feedback and support based on the evaluated progress data.

12. The method of claim 11, the method further comprising:
performing, by a given one of a plurality of agents supported by artificial intelligence, an assigned task; and
cooperating, by the given agent, with others of the plurality of agents to generate output.

13. The method of claim 11, wherein the step of evaluating input of the patient comprises evaluating input of the patient using one or more of speech emotion detection, video emotion detection, speech recognition, and user tapping.

14. The method of claim 11, the method further comprising evaluating the input of the patient and delivering the dialogue in real time.

15. The method of claim 11, the method further comprising:
predicting a potential emergency based on the evaluated input; and
triggering an alarm based on the prediction.

16. The method of claim 11, the method further comprising formulating the dialogue based on the evaluated input of the patient.

17. The method of claim 16, the method further comprising tailoring the dialogue based on the patient's medical data, a severity of the patient's dementia, the patient's speech capability, and background information regarding the patient.

18. The method of claim 11, wherein the step of engaging the patient with the application comprises engaging the patient with the application using guided personalized goals.

19. The method of claim 11, wherein the step of logging and evaluating progress data of the patient comprises logging and evaluating progress data of the patient including a progression of dementia over a given period of time.

20. The method of claim 11, wherein the step of operating the application for the patient with dementia, wherein the application comprises at least one applied virtual environment comprises operating an application for a patient with dementia, wherein the applied virtual environment comprises any 3D room including but not limited to a living room, a bedroom, a garden, a barn paddock and pasture, a bathroom, a café, a temple, or a ZOO.
